(19) United States
(12) Patent Application Publication
(10) Pub. No.: US 2015/0047416 A1
(43) Pub. Date: Feb. 19, 2015

(54) Scanned 1-D Gas Plume Profile and Flux Measurements Using Multiple Analysis Instruments

(71) Applicant: Picarro, Inc., Santa Clara, CA (US)

(72) Inventors: Chris W. Rella, Sunnyvale, CA (US); Eric R. Crosson, Livermore, CA (US); David Steele, San Francisco, CA (US); Marc L. Fischer, Berkeley, CA (US)

(21) Appl. No.: 14/532,999

(22) Filed: Nov. 4, 2014

Related U.S. Application Data
(63) Continuation-in-part of application No. 13/934,023, filed on Jul. 2, 2013.
(60) Provisional application No. 61/740,896, filed on Dec. 21, 2012.
(60) Provisional application No. 61/820,926, filed on May 8, 2013.

(51) Int. Cl. G01N 33/00

(52) U.S. Cl. CPC G01N 33/0011
USPC 73/30.01

(57) ABSTRACT

A gas concentration image (i.e., concentration vs. position data) in a cross section through a gas plume is obtained. Such measurements can be obtained by moving a 1D array of gas sample inlets through the gas plume. By combining a gas concentration image with ambient flow information through the surface of the gas concentration image, the leak rate (i.e., gas flux) from the leak source can be estimated. Multiple gas analysis instruments can be employed in connection with sweeping a 1-D array of measurement ports through the gas plume in order to reduce analysis time.

### CROSS REFERENCE TO RELATED APPLICATIONS

This application is a continuation in part of U.S. Ser. No. 13/934,023 filed Jul. 2, 2013, and hereby incorporated by reference in its entirety. U.S. Ser. No. 13/934,023 claims the benefit of U.S. provisional patent application 61/740,896, filed on Dec. 21, 2012, and hereby incorporated by reference in its entirety. U.S. Ser. No. 13/934,023 also claims the benefit of U.S. provisional patent application 61/820,926, filed on May 8, 2013, and hereby incorporated by reference in its entirety.

### FIELD OF THE INVENTION

This invention relates to gas handling for measurement of gas plumes in an ambient.

### BACKGROUND

Methods for detecting gas leaks in ambient air have been investigated for many years. One of the basic problems of such measurements is to determine an estimate of the total gas leak rate from the leak source. A single point gas concentration measurement is not sufficient to determine the total gas leak rate. For example, a single measurement of a high gas concentration could mean the measurement point is very close to a relatively small leak, or some distance away from a large gas leak.

Accordingly, multi-point measurement techniques for gas leak detection have been investigated. U.S. Pat. No. 8,190,376 is a representative example. In this work, two or more gas concentration sensors are disposed in a region of interest, and these concentration measurements are combined with meteorological information (wind speed, direction and stability) to provide estimates of leak rate and leak location. A similar approach is considered in U.S. Pat. No. 6,772,071.

Although this approach can work well for leak detection in a fixed location, e.g., in a chemical plant, it is often necessary to perform leak detection from a mobile platform such as a moving vehicle. One important application of mobile gas leak detection is detecting leaks in natural gas utility distribution systems. For mobile gas leak detection, it is not usually possible to have several gas concentration sensors disposed around the location of possible gas leaks, thereby making the above-described approach inapplicable.

Accordingly, it would be an advance in the art to provide improved gas leak measurements, especially from a mobile platform.

### SUMMARY

The present approach is based on the idea of obtaining a gas concentration image (i.e., concentration vs. position data) in a cross section through a gas plume. Such measurements can be obtained by using a 2D array of gas sample inlets, or by moving a 1D array of gas sample inlets through the gas plume. For example, the 1D array of gas sample inlets could be disposed on a mast affixed to a vehicle. By combining a gas concentration image with ambient flow information through the surface of the gas concentration image, the leak rate (i.e., gas flux) from the leak source can be estimated.

Gas samples can be simultaneously acquired by filling gas sample storage chambers (one gas sample storage chamber for each of the gas sample inlets). This is the default operation mode, which is convenient to regard as recording mode. The other operating mode is a playback mode, where the gas samples in the gas sample storage chamber are sequentially provided to a gas analysis instrument. Triggering from the recording mode to the playback mode can be based on ancillary measurements (e.g., detection of an above baseline gas concentration).

In this manner, the expense of having one gas analysis instrument for each of the measurement points can be avoided. Another advantage of the present approach is that using a single analysis instrument means that cross-calibrating multiple analysis instruments is not required. An important feature of this approach is that it does not require sensors to be disposed around the location of a possible gas leak. Instead, measurements all from one side of the gas leak can suffice, provided the measurement points include a good cross section of the gas plume.

In alternative embodiments, multiple gas analysis instruments are employed to reduce analysis time.

### BRIEF DESCRIPTION OF THE DRAWINGS

FIG. 1A shows a side view of a gas plume.

FIG. 1B shows an end view of the gas plume of FIG. 1A.

FIG. 2 shows an exemplary 2D array of gas sample inlets.

FIG. 3 shows an exemplary 1D array of gas sample inlets configured to be movable through a gas plume.

FIGS. 4A-C show examples of 1D arrays of gas sample inlets mounted on a vehicle.

FIG. 5 shows recording mode of an exemplary embodiment of the invention.

FIG. 6 shows playback mode of an exemplary embodiment of the invention.

FIG. 7 shows an exemplary playback signal.

FIG. 8 shows how the various parts of the signal of FIG. 7 are related to the gas sample inlet ports.

FIG. 9 shows the results of FIGS. 7 and 8 related to a common horizontal position axis.

FIG. 10 shows an example of use of multiple instruments.

FIG. 11 shows results relating to the embodiment of FIG. 10.

FIG. 12 shows an arrangement of multiple inlets coupled to a single gas line.

FIG. 13 shows an arrangement of inlets and connections that provides equal transit time from each inlet to an analysis instrument.

### DETAILED DESCRIPTION

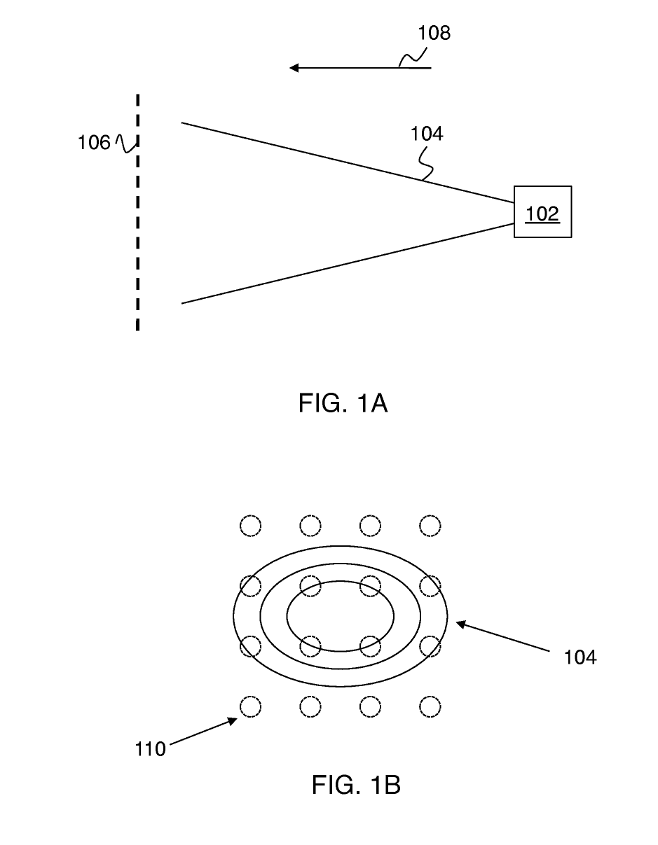

FIG. 1A shows a side view of a gas plume. FIG. 1B shows an end view of the gas plume of FIG. 1A. Here 102 is a source of a gas leak, which leads to a gas plume 104 as driven by an ambient wind 108. A smooth vertical surface 106 intersects the gas plume 104. FIG. 1B shows a view in the plane of surface 106, where measurement points 110 (dotted lines) overlap with the plume 106.

Consider a planar (or other) surface, through which one wants to measure the flux of molecules. The flux of molecules through the plane is given by the following integral:

$$Q ( t ) = {\int }_{A} k ( C ( x , y , t ) - {C}_{0} ) {u}^{->} ( x , y , t ) \cdot \hat{n} d A$$

where C (x,y,t) is the concentration at a given point in space on the surface A at time t, C₀ is the background concentration of the target gas in the ambient, \vec{u} (x,y,t) is the velocity of the gas through the surface, and \hat{n} is the normal to the surface element dA. The constant k converts volumetric flow in m³/s to moles/s, such that the units of emission Q(t) are, for example, moles/second. In this manner, gas concentration image measurements can be related to the total emission Q(t) from the leak source.

As indicated above, and described in greater detail below, gas samples are acquired simultaneously into two or more gas sample storage chambers, and then provided sequentially to a gas analysis instrument. Thus an exemplary method includes the following steps:
1) simultaneously collecting two or more gas samples in two or more gas sample storage chambers, where the gas sample storage chambers receive input gas from two or more measurement locations, and where the measurement locations are configured to be on a smooth vertical surface;
2) providing the two or more gas samples sequentially to a gas analysis instrument to provide gas concentration data points;
3) relating the gas concentration data points to the two or more measurement locations to provide a gas concentration image of the smooth vertical surface; and
4) providing the gas concentration image as an output.

Optionally, the further steps of: 5) obtaining an estimate of ambient flow velocity through the smooth vertical surface; and 6) computing a gas flux estimate from the gas concentration image and the estimate of ambient flow velocity can be performed. The flow velocity estimate can be a single speed and direction estimate, or it can account for variation in speed and/or direction as a function of height above ground. When a height-dependent wind speed is used, the functional form of the wind speed vs. height can be either a fixed functional form, or a form based upon real-time conditions, such as wind speed, solar radiation, terrain, or other atmospheric conditions.

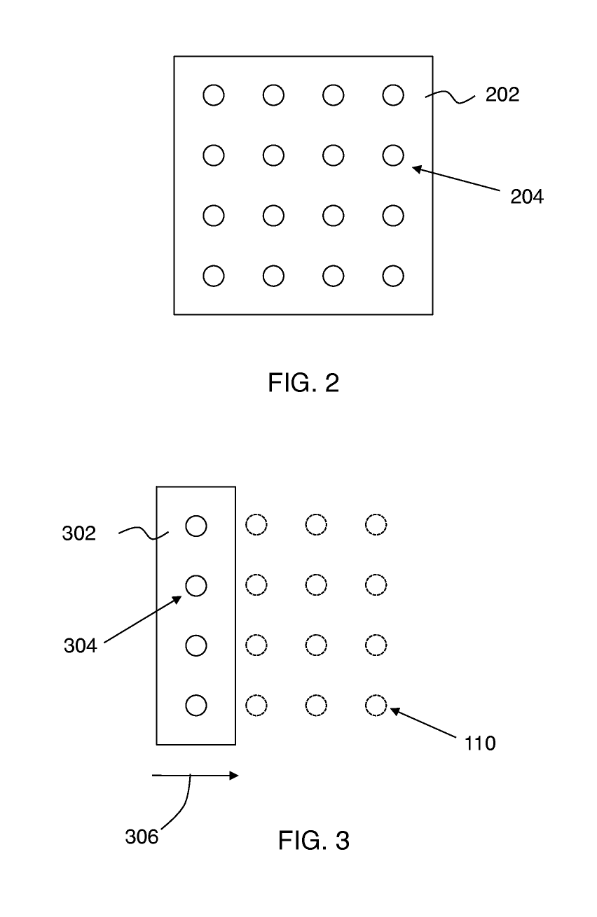

The measurement locations for gas concentration images can be defined in various ways. One way is to have a 2D array of measurement ports corresponding to the measurement locations. FIG. 2 shows an exemplary 2D array of measurement ports. In this example, an array of measurement ports 204 is provided in member 202 to define the measurement locations. Such an array could be used as a fixed installation, or it could be disposed on a vehicle.

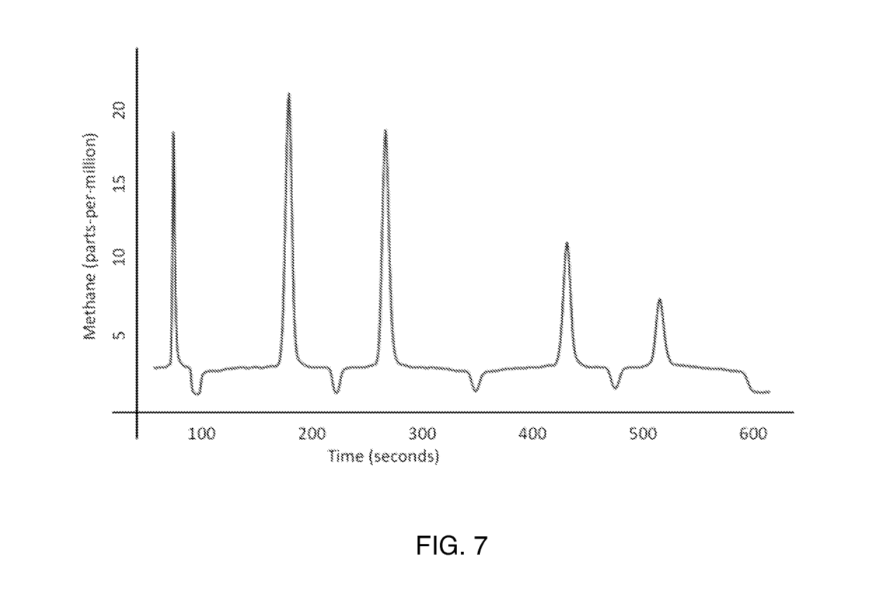

Another approach for defining the measurement locations is to have a 1D array of measurement ports that can be moved through gas plumes to measure them. FIG. 3 shows an exemplary 1D array of measurement ports configured to be movable through a gas plume. In this example, an array of measurement ports 304 is provided in member 302 to define the measurement locations. As member 302 moves as indicated by arrow 306, a 2D array of measurement locations 110 is defined. For simplicity, FIG. 3 shows a discrete 2D array of measurement locations. It is also possible for the gas samples taken via inlets 304 to be measured continuously, in which case the resulting gas concentration image is still 2-dimensional, but the measurement locations are a set of lines instead of discrete points. This approach is considered in the example of FIGS. 7-9. The resulting gas concentration image is a snapshot in time, to the extent that the transit time of member 302 through the plume is substantially faster than the evolution of the plume in time. Possible evolution of the plume during transit of the vehicle through the plume can lead to over- or under-estimates of the emissions.

In general, the measurement locations can be an array (either Cartesian or non-regular spacing) of ambient air measurement points, distributed on a surface substantially orthogonal to the wind direction. A measurement point can be anything that defines the location of the gas being sampled in a point, line and/or area, such as an inlet of a tube (point), an array of tube inlets (points) where the flow from the inlet ports are combined into a single tube, tubes with slots in the side walls (line), and general apertures (area).

When a 1-D array of measurement ports is used to determine a gas concentration image, it is important for the gas concentration measurements to include time information, and to relate the measurement times to measurement positions.

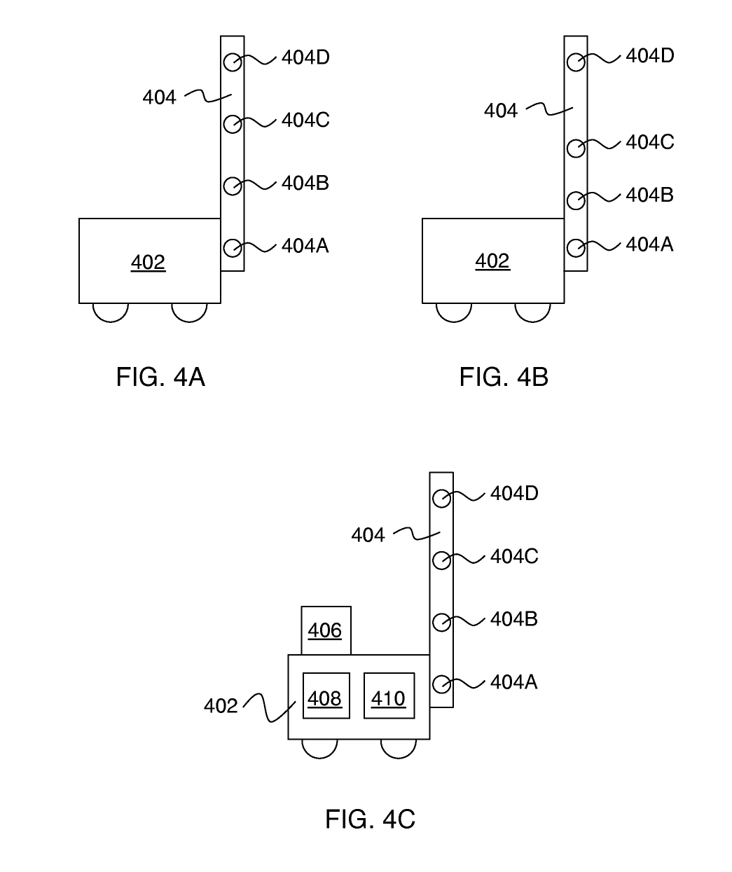

One approach for providing a 1D array of measurement ports that can readily move through a gas plume is to affix a mast to a vehicle. FIG. 4A shows an example, where mast 404 is affixed to vehicle 402. Mast 404 includes measurement ports 404A, 404B, 404C and 404D. This example shows four measurement ports. In general, two or more measurement ports can be employed. The measurement locations are defined by the 1-D array of measurement ports disposed on mast 404 that sweep out a 2-D array of measurement points (discrete points, or a set of continuous lines) as the vehicle moves.

Optionally, the locations of the measurement ports on the mast can be altered during operation and/or adjusted between measurement runs. FIG. 4B shows an example, where the measurement ports on FIG. 4B are in different locations than in the example of FIG. 4A. Optionally, the arrangement of the measurement ports could be changed using a servo system to toggle between two or more pre-determined measurement port configurations, or to a configuration where one or more of the measurement ports moves dynamically during the measurement. Optionally, the configuration of measurement ports can include vertically separated measurement ports and one or more additional low-height measurement ports near ground level that can be used specifically to identify on-road below-the-vehicle leaks that are not sufficiently offset from the vehicles axis of motion. It is useful to identify below-the-vehicle leaks, because a leak that is too close to the vehicle's axis of motion will lead to a plume that is not well-formed by the time it is intercepted by the vertical array of measurement points, and the emission rate measurement may be unreliable. The near ground vertical concentration gradient can be used to identify below-vehicle leaks. The near ground horizontal (transverse to the vehicle motion) gradient can also be used to identify below-vehicle leaks.

Optionally, further instrumentation can be included on the vehicle 402. FIG. 4C shows an example, where 406 is an instrument for measuring ambient flow velocity, 408 is a global positioning system (GPS) receiver to track horizontal position of the vehicle, and 410 is a vehicle speed sensor and a subsystem for converting time and vehicle speed information to position information. Ambient flow instrument 406 can include a system to relate on-board wind speed and direction measurements to ambient wind speed and direction by accounting for vehicle speed and direction. The on-board wind velocity (i.e., speed and direction) is the vector sum of the ambient wind velocity and the vehicle velocity. It is also possible to include any subset of ambient flow instrument 406, GPS 408 and vehicle speed sensor and subsystem 410. Wind speed and direction can also be obtained from a nearby fixed instrument (e.g., from publicly available weather information). More generally, wind speed, time of day, solar radiation, atmospheric turbulence, or other atmospheric measurements either on the vehicle or nearby can be used to further improve measurement accuracy.

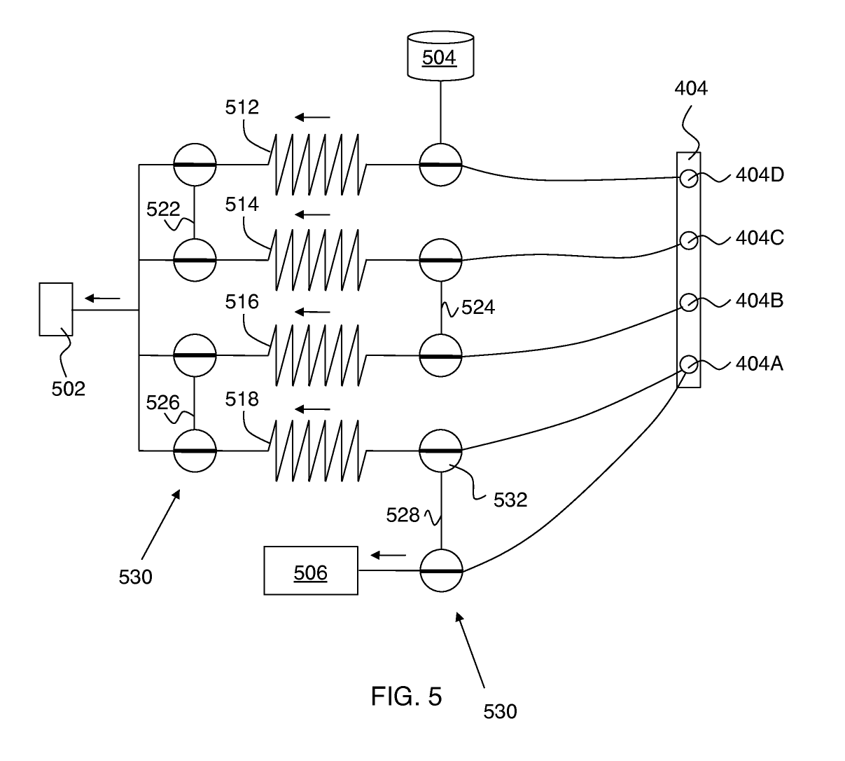

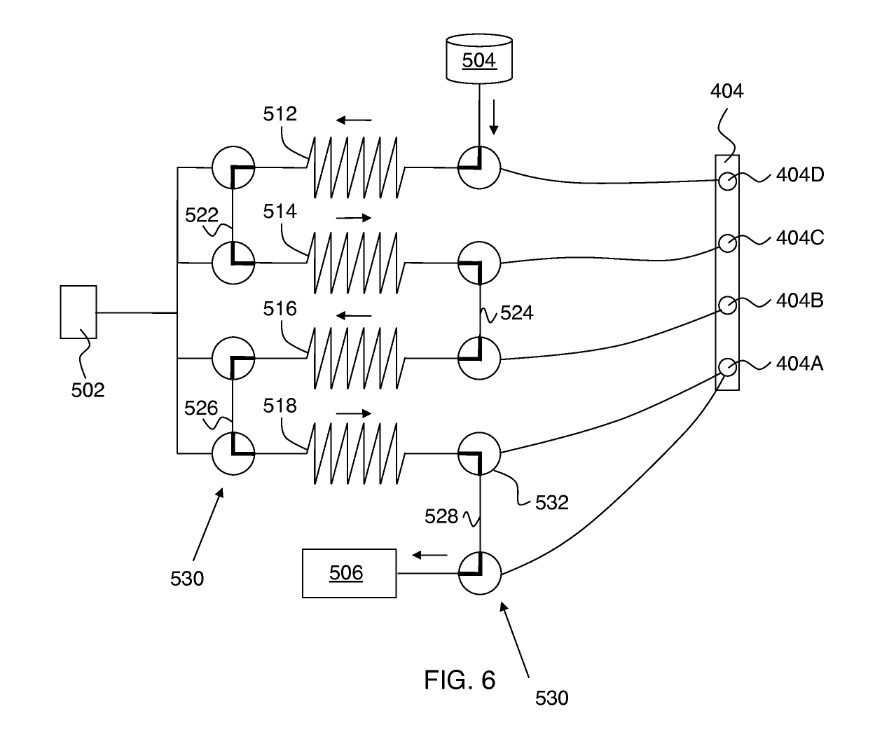

FIGS. 5 and 6 show an exemplary embodiment of the invention in recording mode (FIG. 5) and playback mode (FIG. 6). In this example, mast 404 includes measurement ports 404A, 404B, 404C, and 404D. In recording mode

(FIG. 5), pump 502 simultaneously draws gas samples from measurement ports 404A, 404B, 404C, and 404D into gas sample storage chambers 518, 516, 514, and 512, respectively. Gas flow control manifold 530 (which includes nine three-way valves, one of which is referenced as 532) is configured to allow this flow, as shown by the heavy lines in the three way valves. Mast 404 is affixed to a vehicle, so the measurement locations defined by the measurement ports are on a smooth vertical surface.

A gas analysis instrument 506 is included, and it receives gas from one of the measurement ports (404A in this example). Any kind of gas analysis instrument can be used. Preferred instruments include cavity enhanced optical spectroscopy instruments, such as cavity ring down spectroscopy (CRDS) instruments and cavity enhanced absorption spectroscopy (CEAS) instruments. In recording mode, gas analysis instrument 506 is mainly used to trigger the switch into playback mode. Any suitable way to trigger playback mode can be used, and practice of the invention does not depend critically on these details (e.g., which of the measurement ports instrument 506 is connected to in recording mode). Optionally, instrument 506 can be used to measure one or more of the measurement ports in real time during the recording phase to ensure that the measurement surface is substantially downwind of the source of emissions.

In playback mode (FIG. 6) the gas flow control manifold 530 is configured to provide the two or more gas samples sequentially to the gas analysis instrument to provide gas concentration data points. This is shown by the heavy lines in the three way valves on FIG. 6.

The system is configured to relate the gas concentration data points to the two or more measurement locations to provide a gas concentration image of the smooth vertical surface, as described above.

More generally, outputs from this measurement can include one or more of the following: 1) An estimate of the emissions transported by the wind through the surface defined by the measurement points, either averaged over the time period of the measurement, or reported with the time resolution of the device, determined by a) the response time of the instrument, b) the ratio of flows between recording and playback, and c) time dispersion of gas in the tubes during recording and playback; 2) An image of the concentration measured on the surface, averaged over the time period of the measurement; or 3) A video of the concentration measured on the surface, as it evolved during the time period of the measurement.

The number of gas sample storage chambers is limited only by the number of 3-way valves (two are needed per gas sample storage chamber), the speed of the analysis instrument, the desired duty factor of the measurement, and the potential for pulse spreading within the tubing (which is negligible for most practical situations). Pulse spreading is likely to be most serious during high flow rate playback.

Preferably, the gas sample storage chambers are configured as tubes having a length to diameter ratio of 20:1 or more (more preferably 100:1 or more). This high aspect ratio usefully provides a time axis for gas samples in the gas sample storage chambers. Further details on this concept of preserving a time axis in gas samples in narrow tubes are given in U.S. Pat. No. 7,597,014, filed Aug. 15, 2006, and hereby incorporated by reference in its entirety.

Optionally, plumbing manifold 530 can include volumetric or mass flow sensors located on each of the recording lines and/or the analysis line, so that accurate time reconstruction is possible given the valve switching times and the molar volumes contained in the gas manifold and connection tubing. This can make the system more robust to unexpected conditions (pressures, flow conductivity, etc.) in the plumbing system.

For time efficiency, the flow rate through the gas analysis instrument during playback mode is preferably larger than the flow rate through the gas sample storage chambers during recording mode. Precise flow sensing or control can be used to maintain the integrity of the time axis for the several gas samples, and to make sure that all of the gas sample storage chambers are filled with gas that corresponds to the same period of time.

It is preferred for the system to include a push gas source 504, as shown on FIGS. 5 and 6. Provision of a predetermined push gas during playback, as opposed to just allowing ambient in during playback can provide significant advantages. The most important advantage is the ability to use the push gas to identify the transitions between the various gas samples being analyzed in playback mode. This can be accomplished by having the push gas separate the gas samples when they are provided to the gas analysis instrument 506. For example, trapped push gas in tubing 522, 524, 526 and 528 can provide such separation. Push gas can be trapped in these sections of tubing by performing a complete playback of all gas samples until push gas is the only gas present in the system. After that, switching to recording mode (FIG. 5) will trap push gas in tubing 522, 524, 526 and 528. The push gas is preferably distinguishable from the gas samples using results from the gas analysis instrument.

The push gas can be distinguished from the gas samples by having a different concentration of the primary gas (i.e., the gas which is being measured in ambient) than is possible in the gas samples and/or by including a secondary gas species which the gas analysis instrument is responsive to and which is not expected to occur in the gas samples. The optional use of a secondary gas species in the push gas can avoid disrupting the primary measurement by changing concentration of the primary species in the push gas.

Optionally, the push gas concentration can be below ambient concentration levels, so that this low signal is unique to the push gas and will not exist under reasonable conditions in the recorded ambient gas, thus giving a clear signature for identification of the timing pulses provided by the push gas. Optionally, zero air (i.e., ambient air filtered to contain less than 0.1 ppm total hydrocarbons) can be the push gas, or zero air can be used to dilute ambient air to provide the push gas. Optionally, the component of the push gas used to provide the timing information can be CO₂. Optionally, the push gas can be ambient air that is subsequently treated by a sodalime, ascarite, or other CO₂ trap to reduce the CO₂ concentration below ambient levels.

Optionally, the push gas concentration can be above ambient concentration levels. Optionally, a high concentration of the push gas species can be contained in a semipermeable container, such as a section of PTFE (polytetrafluoroethylene) tubing, such that slow diffusion of the gas from the container into a sample of ambient air provides the push gas for timing measurement.

The example of FIGS. 5 and 6 shows providing the two or more gas samples sequentially to the gas analysis instrument by connecting the gas sample storage chambers to each other in series and flowing the gas samples to the gas analysis instrument. Alternatively, the two or more gas samples can be provided sequentially to the gas analysis instrument by sequentially switching the gas sample storage chambers to flow to the gas analysis instrument (e.g., with an N-way valve for N gas sample storage chambers). As another alternative, banks of measurement ports can be measured serially, with different banks being selected by a multi-position valve at the inlet of instrument 506. Such use of banks of measurement ports can mitigate the gas dispersion in the gas sample storage chambers, because the sample in the last gas sample storage chamber (e.g., 512 on FIG. 6) does not need to be transported through all the other gas sample storage chambers before it reaches the measurement instrument.

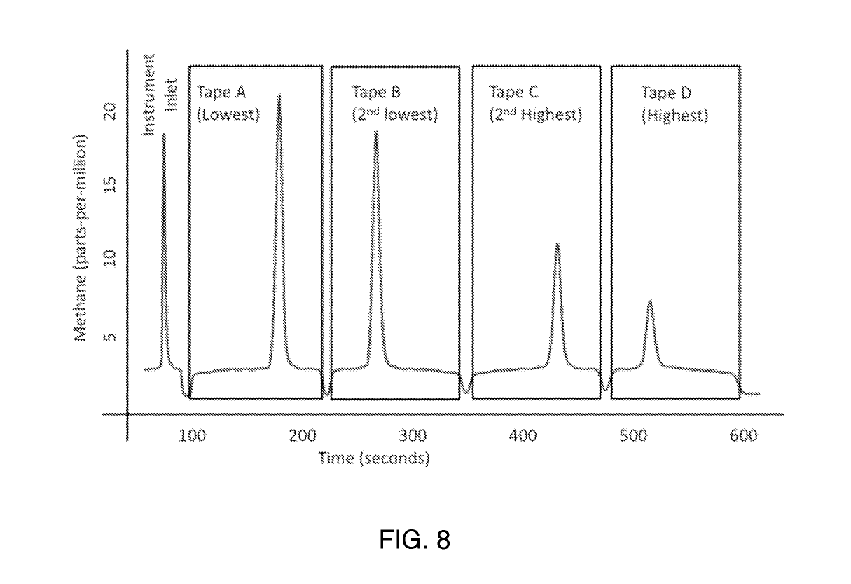

Important features of the present approach can be better appreciated by considering the data of FIGS. 7-9. FIG. 7 shows a playback signal from a 4-channel system as in FIGS. 5 and 6. In this exemplary system, gas sample storage chambers 512, 514, 516, and 518 have capacity 500 scc (standard cubic centimeter), and the recording flow rate is 1000 sccm (standard cubic centimeters per minute). Thus, the gas sample storage chambers each provide 30 seconds of stored gas sample. FIG. 8 shows how the various parts of the signal of FIG. 7 are related to the gas sample inlet ports. Here Tape A relates to measurement port 404A, Tape B relates to 404B, etc. The dips in the measured concentration are from trapped push gas that separates the samples. Here it is clear that the time axis for Tape B is reversed relative to the time axis for Tape A, which is consistent with the opposite flow directions through gas sample storage chambers 516 and 518 shown on FIG. 6. Similarly, the time axis of Tape D is reversed with respect to Tape C, while Tape A and Tape C have consistent time axes. All of this is consistent with the flow directions through gas sample storage chambers 512, 514, 516, and 518 on FIG. 6.

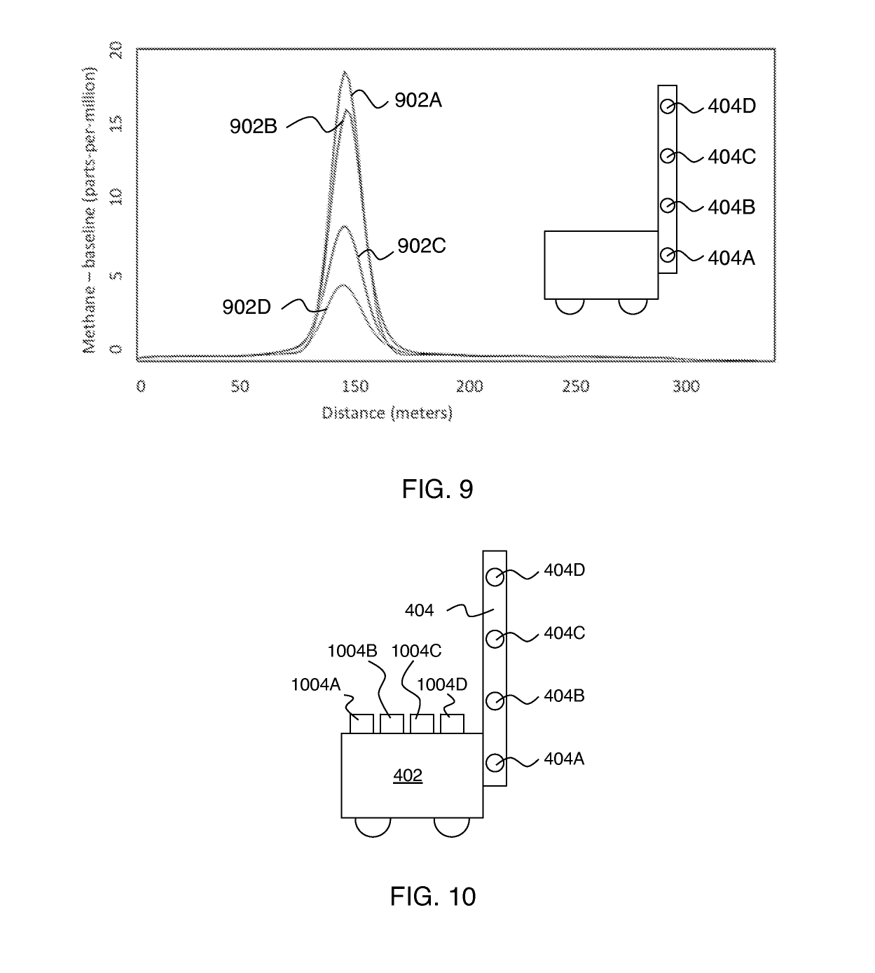

FIG. 9 shows the results of FIGS. 7 and 8 where time has been converted to position, thereby relating the four gas samples to a common horizontal position axis. This information can be used to provide a 2D gas concentration image for the plume, which in this example gave a source flux estimate of 1.5±0.3 L/s based on a wind speed estimate of 2.5 m/s (normal to the measurement surface) and a vehicle speed of 10.8 m/s.

The preceding embodiments has all related to the use of a single gas analysis instrument. Although such configurations are often preferred to minimize cost, there are also cases where the use of multiple instruments is preferred. FIG. 10 shows an exemplary embodiment that is similar to the embodiment of FIG. 4A, except that on-board instruments 1004A, 1004B, 1004C and 1004D are connected to (and correspond to) measurement ports 404A, 404B, 404C and 404D respectively. For simplicity, these connections are not shown on the figure. The above-described variations relating to single-instrument embodiments are also generally applicable to multi-instrument embodiments. In cases where multiple instruments are employed, the number of instruments can be the same as the number of measurement ports (e.g., as shown on FIG. 10), or it can be less than the number of measurement ports. In cases where there are fewer instruments than ports, the above-described recording and playback approaches can be applied to couple multiple ports to one or several of the gas analysis instruments. In all cases, use of multiple gas analysis instruments allows analysis of the gas samples to proceed in parallel, thereby reducing analysis time.

Using an independent concentration analyzer for each independent inlet is generally prohibitive from the standpoint of cost and complexity for a 2D array of measurement points, but it can be practical for a 1D array of inlets swept rapidly through the gas plume. In some situations, the cost of the additional measurement instruments is offset by the advantages; i.e., 1) a faster measurement of the emissions (taking only as long as it takes to traverse the plume), and 2) the ability to measure emissions continuously without the need to interrupt the measurement for ‘playback’ from gas storage containers. This second advantage is important where there are many emissions sources in close physical proximity, such as in and around natural gas distribution systems in cities, when interrupting the measurement is a significant disadvantage to practical implementation.

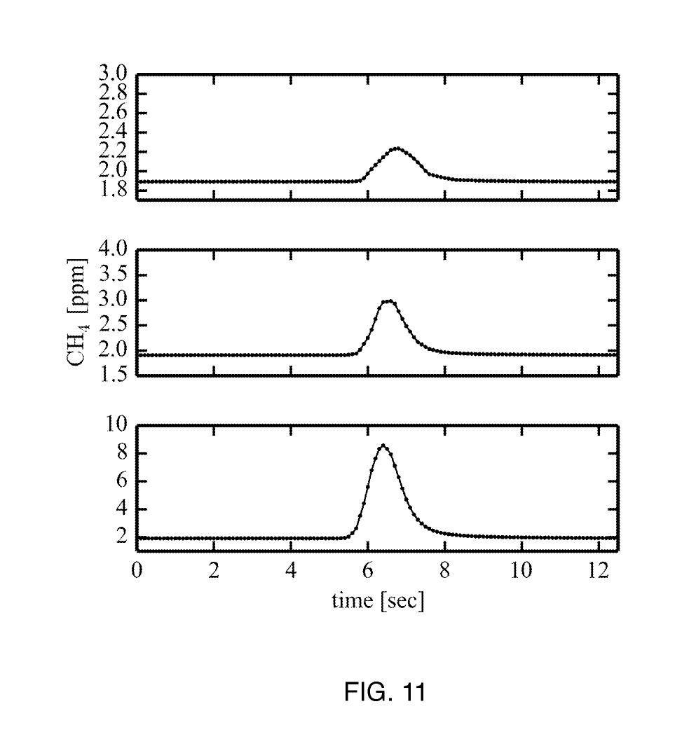

FIG. 11 shows results relating to the embodiment of FIG. 10, with three inlets disposed vertically on a vehicle mast, and with a separate analyzer reporting the methane concentration observed on each inlet. The vehicle path traversed a methane plume approximately 40 ft downwind of a ground level leak. The concentration observed at about 0.5 m, 1.5 m, and 2.5 m above ground level are displayed in the bottom, middle, and top panels, respectively. As expected, there is a strong vertical gradient in the observed concentration, consistent with the propagation of a point source methane leak downwind of the source.

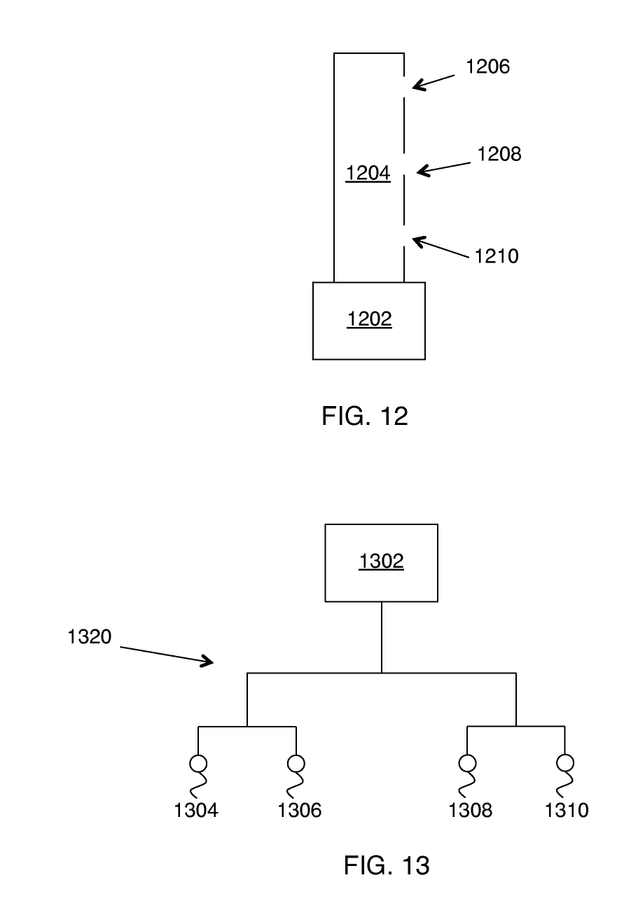

It is well-known that while the time-averaged ensemble of a gas plume is a smoothly varying Gaussian, a fast (<5 second) snapshot of a plume can have a significant degree of internal spatial structure. To resolve that spatial structure, it is preferred to dispose a densely spaced set of gas inlets on the vertical mast, and a separate gas analyzer for each inlet. However, if the goal is emissions quantification rather than plume imaging, it can be acceptable to average the concentration over some substantial vertical distance, thus reducing the number of independent analyzers required. This averaging can be performed automatically by a spatially distributed inlet source, such as an array of points, a line inlet (or inlets), an area inlet (or inlets). If the flow into the distributed inlet is spatially uniform, then the concentration in the combined stream is equal to the average concentration in each of the individual streams, due to conservation of mass. Typically, controlling the flow into an array of point inlets such as small bore tubes (with <0.5″ inner diameter) is easier to practically achieve than other non-point inlets. FIG. 12 shows an example, where several inlets 1206, 1208 and 1210 provide input gas to gas line 1204, which is connected to a single gas analysis instrument 1202. With this arrangement, the input to instrument 1202 is an average of the gas samples at inlets 1206, 1208 and 1210.

It is a further advantage for horizontal spatial reconstruction of the measured plume, and to avoid interference of nearby plumes in the analysis, that the transit time to the instrument of gas presented to different locations of the distributed inlet be the same. The simplest form of array inlets, i.e., a tube perforated periodically or aperiodically by small holes, does not achieve this goal, since gas which enters at the far end of the perforated tube has a significantly longer transit time to the instrument than gas which enters at the near end of the tube. One way to accomplish this goal is to equalize the length of tubing from each of the inlets to the instrument, although there are other, more efficient and practical configurations. FIG. 13 shows an exemplary configuration where inlets 1304, 1306, 1308 and 1310 all have the same transit time to instrument 1302 because of connections 1320 as shown. The basic idea here is to reduce the total amount of tubing required for equal times from each inlet by combining inlets in a generally tree-like configuration of tubing.

1. A method for performing gas concentration measurements in an ambient, the method comprising:
simultaneously collecting two or more gas samples from two or more measurement locations, wherein the measurement locations are configured to be on a smooth vertical surface, and wherein the measurement locations are defined by a 1-D array of measurement ports that is moved along the smooth vertical surface;
providing the two or more gas samples to one or more gas analysis instruments to provide gas concentration data points;
relating the gas concentration data points to the two or more measurement locations to provide a gas concentration image of the smooth vertical surface; and
providing the gas concentration image as an output.

2. The method of claim 1, wherein the gas concentration data points include measurement time, and further comprising relating measurement time to measurement position to provide the gas concentration image.

3. The method of claim 1, further comprising:
obtaining an estimate of ambient flow velocity through the smooth vertical surface; and
computing a gas flux estimate from the gas concentration image and the estimate of ambient flow velocity.

4. The method of claim 3, wherein the estimate of ambient flow velocity through the smooth vertical surface is provided as a function of height above ground.

5. The method of claim 1, wherein two or more gas analysis instruments are provided for analysis of the gas samples in parallel.

6. A system for performing gas concentration measurements in an ambient, the system comprising:
a gas flow control manifold configured to simultaneously collect two or more gas samples from two or more measurement locations, wherein the measurement locations are configured to be on a smooth vertical surface, and wherein the measurement locations are defined by a 1-D array of measurement ports that is moved along the smooth vertical surface; and
one or more gas analysis instruments;
wherein the gas flow control manifold is configured to provide the two or more gas samples to the gas analysis instruments to provide gas concentration data points;
wherein the system is configured to relate the gas concentration data points to the two or more measurement locations to provide a gas concentration image of the smooth vertical surface.

7. The system of claim 6, further comprising a mast configured to be affixed to a vehicle, wherein the 1-D array of measurement ports is disposed on the mast.

8. The system of claim 7, wherein positions of the measurement ports on the mast can be altered in operation.

9. The system of claim 6, further comprising instruments for measuring ambient flow velocity.

10. The system of claim 7, further comprising a global positioning system (GPS) receiver to track horizontal position of the vehicle.

11. The system of claim 7, further comprising a vehicle speed sensor and a subsystem for converting time and vehicle speed information to position information.

12. The system of claim 6, wherein two or more gas analysis instruments are provided for analysis of the gas samples in parallel.

13. The system of claim 6, wherein a selected two or more of the measurement ports are connected to a selected one of the gas analysis instruments.

14. The system of claim 13, wherein the selected measurement ports are connected to the selected gas analysis instrument such that transit times from each of the selected measurement ports to the selected gas analysis instrument are substantially equal.
